(19) United States
(12) Patent Application Publication
(10) Pub. No.: US 2016/0367196 A1
(43) Pub. Date: Dec. 22, 2016

(54) APPARATUS FOR MEASURING BIOMETRIC INFORMATION, APPARATUS FOR MEASURING BODY TEMPERATURE, AND ELECTRONIC DEVICE HAVING THE APPARATUSES

(71) Applicant: Samsung Electronics Co., Ltd., Gyeonggi-do (KR)

(72) Inventors: Yong-Yi KIM, Gyeonggi-do (KR); Jae-Kwang SIM, Gyeonggi-do (KR); Kyung-Seok KIM, Gyeonggi-do (KR)

(21) Appl. No.: 15/184,189

(22) Filed: Jun. 16, 2016

(30) Foreign Application Priority Data
Jun. 17, 2015 (KR) 10-2015-0085928

(51) Int. Cl. A61B 5/00; A61B 5/0408; A61B 5/0404; A61B 5/0205; A61B 5/01

(52) U.S. Cl. CPC A61B 5/6898; A61B 5/02055; A61B 5/01; A61B 5/0404; A61B 5/4872; A61B 5/04085; A61B 5/7475; A61B 5/742; A61B 5/0022; A61B 5/02416

(57) ABSTRACT

An apparatus for measuring biometric information includes a cover, a sensor provided inside the cover, for measuring biometric information, and an electrode assembly provided outside the cover, and having a guide therein in at least partial alignment with a cover opening. The arrangement of the electrode assembly on the outside of the cover provides a measurement distance for measuring the biometric information.

### CLAIM OF PRIORITY

This application claims the benefit of priority under 35 U.S.C. §119(a) of a Korean patent application filed in the Korean Intellectual Property Office on Jun. 17, 2015 and assigned Serial No. 10-2015-0085928, the entire disclosure of which is incorporated herein by reference.

### BACKGROUND

Field of the Disclosure

The present disclosure relates to an apparatus for measuring biometric information about a user.

Description of the Related Art

There are a number of biometric information meters that are adapted to various body conditions. One type of biometric information meter is a body temperature meter. The body temperature meter (i.e. a body thermometer) may operate in a contact manner or contactless manner. A contact body thermometer measures the temperature of a bodyr using, for example, alcohol or mercury. As the contact body thermometer is inserted into an orifice of the body, or contacts an ear, a mouth, or the anus of the user to measure body temperature, the user may feel uncomfortable. Therefore, contactless body thermometer are now more popular than ever before as this technology is being developed.

With regard to the development of a contactless body temperature meter (thermometer), an infrared (IR) body thermometer meter has been developed and commercialized. For example, the IR body thermometer includes a sensor unit, a display, and measurement buttons. The sensor unit measures body temperature by receiving weak IR light generated from the temporal arteries during scanning at a predetermined distance from the surface of the forehead of a user for a predetermined time. The display displays the body temperature measured by the sensor unit as a numeral value.

However, if the IR body thermometer is not sufficiently close to the forehead of the user, it may not normally receive weak IR light generated from the temporal arteries of the forehead, thereby causing an error in body temperature measurement.

In other words, the conventional contactless body thermometer is capable of accurately measuring body temperature only when the senor unit is disposed at the outermost side of the contactless body thermometer and is located within a predetermined distance from the forehead of the user in order to increase the performance and accuracy of body temperature sensing. Such a predetermined distance often includes conjecture on the part of the person collecting the biometric information,

Moreover, due to electrostatic discharge (ESD) of the sensor unit, the performance of the contactless body thermometer can be degraded.

Moreover, in the conventional contactless body thermometer, the sensor unit is installed in a non-isothermal cap, which as a result can introduce an error signal generated from the cap that decreases the accuracy of body temperature measurement.

Consequently, there is a need in the art for a separate mechanical apparatus that can be provided at a predetermined distance from a user without being disposed at the outermost side of a terminal, in order to permit more accurate measurement by a sensor unit.

The above information is presented as background information only to assist with an understanding of the present disclosure. No determination has been made, and no assertion is made, as to whether any of the above might be applicable as prior art with regard to the present disclosure.

### SUMMARY

An aspect of the present disclosure is to address at least some of the above-mentioned problems and/or disadvantages discussed herein and to provide at least the some of the advantages described below. Accordingly, an aspect of the present disclosure is to provide a biometric information measuring apparatus having an electrode unit (electrode) which is disposed outside a cover unit (cover) and secures a measurement distance for measuring biometric information about a user, for accurately measuring a biometric state of the user. The measured distance is “secured” in that the electrode is designed for contact with a person whom the biometric information is to be measured. A guide which may be provided in the electrode permits the signal, for example, from an IR source be a predetermined distance from the patient and provide a more accurate reading than, for example, conventional body temperature apparatus in which a user may judge the appropriate distance from the body to be measured.

Another aspect of the present disclosure is to provide a biometric information measuring apparatus having an electrode assembly which, outside a cover, guides infrared light generated from the body of a user and secures a predetermined measurement distance for measuring the body temperature of the user, for increasing the accuracy of body temperature measurement in a product by preventing introduction of a conventional error signal into a sensor unit (sensor) that measures the body temperature and securing the measurement distance, and increasing the performance of the product by preventing generation of conventional electrostatic discharge (ECD).

In accordance with an aspect of the present disclosure, there is provided an apparatus for measuring biometric information. The apparatus may include a cover, a sensor assembly provided inside the cover the sensor measures biometric information, and an electrode assembly arranged external to the cover. The electrode forms a measurement distance for measuring the biometric information.

In accordance with another aspect of the present disclosure, there is provided an apparatus for measuring body temperature. The apparatus may include a cover having an opening, a printed circuit board provided inside the cover and having a sensor facing the opening of the cover a lens assembly provided in the opening to concentrate infrared light generated from the body of a user onto the sensor, and an electrode assembly provided outside of (external to) the cover. The electrode assembly may include a guide facing the opening, for guiding introduction of the infrared light and forming a measurement distance for measuring a body temperature of the user.

In accordance with another aspect of the present disclosure, there is provided an electronic device having an apparatus for measuring body temperature. The electronic device may include a mounting portion provided on a rear surface of an exterior member of the electronic device, a cover having an opening and provided in the mounting portion, a printed circuit board provided inside the cover and having a sensor unit facing the opening, a lens assembly provided in the opening to concentrate infrared light generated from the body of a user onto the sensor, and an electrode assembly provided outside of the cover. The electrode assembly may include a guide facing the opening, for guiding introduction of the infrared light and forming a measurement distance for measuring a body temperature of the user.

Other aspects, advantages, and salient features of the disclosure will become better-understood to those skilled in the art from the following detailed description, which, taken in conjunction with the annexed drawings, discloses exemplary embodiments of the disclosure.

### BRIEF DESCRIPTION OF THE DRAWINGS

The above and other aspects, features and advantages of certain exemplary embodiments of the present disclosure will be more apparent from the following description taken in conjunction with the accompanying drawings, in which:

FIG. 1 is a front perspective view illustrating an electronic device according to various embodiments of the present disclosure;

FIG. 2 is a rear perspective view illustrating an electronic device according to various embodiments of the present disclosure;

FIG. 3 is a block diagram illustrating a network environment including an electronic device according to various embodiments of the present disclosure;

FIG. 4 is a side sectional view illustrating a structure of an apparatus for measuring body temperature according to various embodiments of the present disclosure;

FIG. 5 is a front view illustrating an apparatus for measuring body temperature, provided on the rear surface of an electronic device according to various embodiments of the present disclosure;

FIG. 6 is a sectional view taken along line A-A′ illustrated in FIG. 5;

FIG. 7 is a front view illustrating an apparatus for measuring body temperature, provided on the front surface of an electronic device according to various embodiments of the present disclosure;

FIG. 8 is a side view illustrating an apparatus for measuring body temperature, provided on one side surface of an electronic device according to various embodiments of the present disclosure;

FIG. 9 is a side view illustrating an apparatus for measuring body temperature, provided on another surface of an electronic device according to various embodiments of the present disclosure;

FIG. 10 illustrates an operation of an apparatus for measuring body temperature provided in an electronic device according to various embodiments of the present disclosure;

FIG. 11 is a flowchart illustrating an operational example of a method for measuring body temperature in an apparatus for measuring body temperature provided in an electronic device according to various embodiments of the present disclosure; and

FIG. 12 is a flowchart illustrating an operational example of a method for measuring an electrocardiogram in an apparatus for measuring body temperature provided in an electronic device according to various embodiments of the present disclosure.

Throughout the drawings, like reference numerals will be understood to refer to like parts, components, and structures.

### DETAILED DESCRIPTION

Terms used in various embodiments of the present disclosure will first be described in brief, followed by a detailed description of various embodiments of the present disclosure.

Although terms used in various embodiments of the present disclosure are general terms selected in consideration of their functions in various embodiments of the present disclosure, they may vary according to the intent of a user or an operator, the customs, or emergence of new technologies. In certain cases, there are terms that the applicant has arbitrarily selected, and their meanings will be described in detail in the following description. Therefore, the terms used in various embodiments of the present disclosure should be defined by the appended claims and their equivalents, not by their names.

The term as used in the present disclosure, ‘first’ or ‘second’ may modify the names of various components, without limiting the components. These expressions may be used to distinguish one component from another component for clarity. For example, a first component may be referred to as a second component and vice versa without implication of an order, and such terminology does not departing the scope of the present disclosure.

Now, a description will be given of an apparatus for measuring biometric information about a user in an electronic device according to various embodiments of the present disclosure. Non-limiting examples of the electronic device according to an embodiment may include all mobile communication terminals operating in conformance to communication protocols of various mobile communication systems, all information and communication devices, multimedia devices, and their application devices, such as a video phone, an e-book reader, a laptop personal computer (PC), a netbook computer, a personal digital assistant (PDA), a portable multimedia player (PMP), an MPEG-1 audio layer-3 (MP3) player, a mobile medical equipment, a camera, or a wearable device (for example, a head-mounted device (HMD) like electronic glasses, electronic clothes, an electronic necklace, an electronic appcessory, an electronic tattoo, or a smart watch).

According to some embodiments, an electronic device may be a home appliance. For example, the home appliance may be at least one of, for example, a television (TV), a digital versatile disk (DVD) player, an audio player, a refrigerator, an air conditioner, a vacuum cleaner, an oven, a microwave oven, a washer, an air purifier, a set-top box, a TV box (for example, Samsung HomeSync™, Apple TV™, Google TV™ or the like), a game console, an electronic dictionary, an electronic key, a camcorder, or an electronic picture frame.

According to some embodiments, an electronic device may be at least one of a medical device (for example, a magnetic resonance angiography (MRA) device, a magnetic resonance imaging (MRI) device, a computed tomography (CT) device, an imaging device, an ultrasonic device, or the like), a navigation device, a global positioning system (GPS) receiver, an event data recorder (EDR), a flight data recorder (FDR), an automotive infotainment device, a naval electronic device (for example, a naval navigation device, a gyrocompass, or the like), an avionic electronic device, a security device, an in-vehicle head unit, an industrial or consumer robot, an automatic teller machine (ATM) in a financial facility, or a point of sales (POS) device in a shop.

According to some embodiments, an electronic device may be at least one of furniture, part of a building/structure, an electronic board, an electronic signature receiving device, a projector, and various measuring devices (for example, water, electricity, gas or electro-magnetic wave measuring devices).

According to various embodiments, an electronic device having a plurality of built-in parts may be one or a combination of two or more of the foregoing devices. According to various embodiments, an electronic device may be a flexible device. In addition, it will be apparent to one having ordinary skill in the art that an electronic device according to various embodiments of the present disclosure is not limited to the foregoing devices.

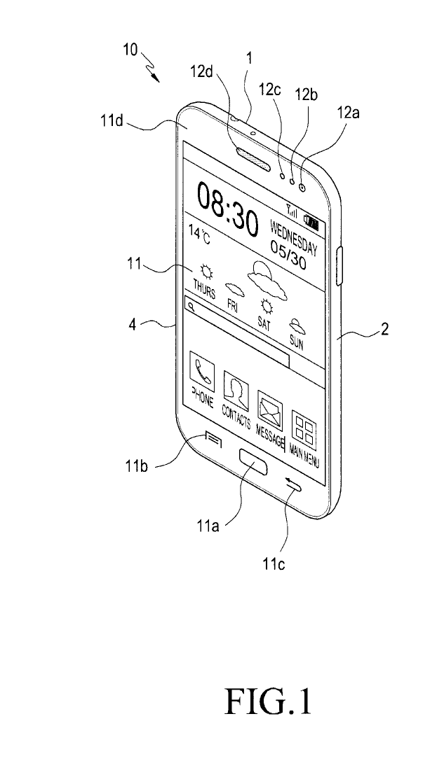

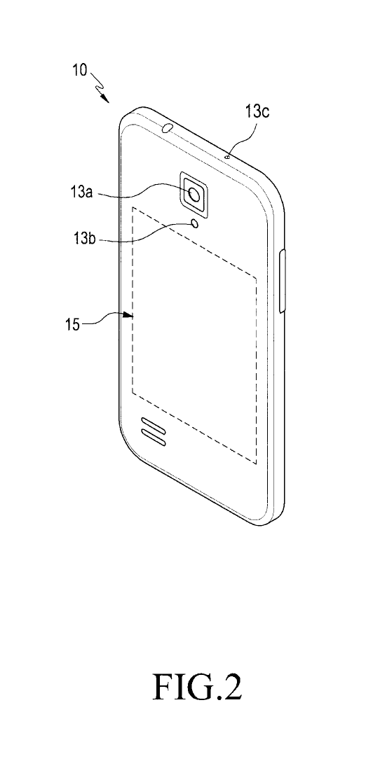

FIG. 1 is a front perspective view of an electronic device, and FIG. 2 is a rear perspective view of the electronic device. In FIGS. 1 and 2, an electronic device 10 may be a smartphone or a wearable device. With reference to FIGS. 1 and 2, components of the electronic device 10 such as a smartphone will be described below.

Referring now to FIGS. 1 and 2, a touch screen 11 may be disposed at the center of the front surface of the electronic device 10, occupying almost the entirety of the front surface of the electronic device 10. In FIG. 1, a main home screen is displayed on the touch screen 11, by way of example. The main home screen is the first screen to be displayed on the touch screen 11, when the electronic device 10 is powered on. In the case where the electronic device 10 has different home screens on a plurality of pages, the main home screen may be the first of the home screens on the plurality of pages. Shortcut icons for executing frequently used applications, a main menu switch key, the time, the weather, and the like may be displayed on the home screen. The main menu switch key may display a menu screen on the touch screen 11. Also, a status bar 11d may be displayed at the top of the touch screen 11 in order to indicate states such as a battery charged state, a received signal strength, and a current time. A home key 11a, a menu button 11b, and a back button 11c may be formed at the bottom of the touch screen 11.

The home key 11a may be used to display the main home screen on the touch screen 11. For example, upon touching of the home key 11a while any home screen other than the main home screen or a menu screen is displayed on the touch screen 11, the main home screen may be displayed on the touch screen 11. Upon touching of the home key 11a during execution of applications on the touch screen 11, the main home screen may be displayed on the touch screen 11. The home key 11a may also be used to display recently used applications or to display a task manager on the touch screen 11. The menu button 11b may provide link menus available on the touch screen 11. The link menus may include, for example, a widget adding menu, a background changing menu, a search menu, an edit menu, an environment setting menu, and the like. The back button 11c may be used to display a screen executed previously to a current screen or end the latest used application.

According to various embodiments of the present disclosure, a first camera 12a, an illumination sensor 12b, or a proximity sensor 12c may be included at a top end of the front surface of the electronic device 10, whereas a second camera 13a, a flash 13b, or a speaker 13c may be included on the rear surface of the electronic device 10. If a battery pack is detachably attached to the electronic device 10, the bottom surface of the electronic device 10 may be a detachable battery cover 15.

The electronic device 10 which will be described below may be any of the aforementioned wearable device, laptop computer, netbook computer, smartphone, tablet PC, Galaxy Tab, and iPad. In the embodiment, the electronic device 10 may be a smartphone.

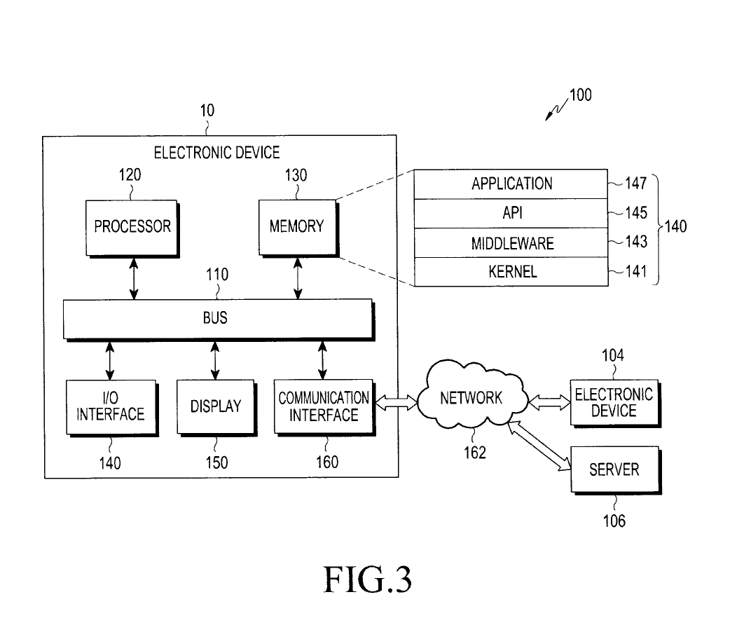

FIG. 3 is a block diagram illustrating a network environment 100 including the electronic device 10 according to various embodiments of the present disclosure.

Referring now to FIG. 3, the electronic device 10 may include a bus 110, at least one processor 120, a non-transitory memory 130, an input/output (I/O) interface 140, a display 150, and a communication interface 160. The bus 110 may be a circuit that interconnects components of the electronic device 10 and allows communication (for example, control messages) between the components.

The at least one processor 120, which comprises hardware such as integrated circuitry configured for operation, may, for example, receive commands from other components (for example, the memory 130, the I/O interface 140, the display 150, and the communication interface 160) through the bus 110, interpret the received commands, and execute computation or data processing related to the interpreted commands.

The non-transitory memory 130 may store commands or data received or generated from the at least one processor 120 or other components (for example, the I/O interface 140, the display 150, and the communication interface 160). The non-transitory memory 130 may include executable instructions, programming modules, for example, a kernel 141, middleware 143, an application programming interface (API) 145, and/or applications 147. Each programming module may be configured in software, firmware, hardware, or a combination of two or more of them, and would be executed by hardware such as an integrated circuit of a processor or microprocessor.

The kernel 141 may control or manage system resources (for example, the bus 110, the at least one processor 120, or the non-transitory memory 130) that are used in executing operations or functions implemented in other programming modules such as the middleware 143, the API 145, or the applications 147. Also, the kernel 141 may provide an interface for allowing the middleware 143, the API 145, or the applications 147 to access and control or manage individual components of the electronic device 10.

The middleware 143 may serve as a medium through which the kernel 141 may communicate with the API 145 or the applications 147 to transmit and receive data. Also, the middleware 143 may control (for example, scheduling or load balancing) task requests received from the applications 147 by, for example, assigning priority levels for using system resources (the bus 110, the at least one processor 120, or the non-transitory memory 130) of the electronic device 10 to at least one of the applications 147.

The API 145 is an interface through which the applications 147 control functions provided by the kernel 141 or the middleware 143. For example, the AP 145 may include at least one interface or function (for example, a command) for file control, window control, video processing, or text control.

According to various embodiments, the applications 147 may include, for example, a short message service/multimedia messaging service (SMS/MMS) application, an email application, an alarm application, a calendar application, an alarm application, a health care application (for example, an application for measuring an exercise amount or a glucose level), or an environment information application (for example, an application for providing information about atmospheric pressure, humidity, or temperature). Additionally or alternatively, the applications 147 may include an application related to information exchange between the electronic device 10 and an external electronic device (for example, an electronic device 104). The information exchange application may include, for example, a notification relay application for transmitting specific information to the external electronic device or a device management application for managing the external electronic device.

For example, the notification relay application may include a function of transmitting notification information generated from another application (for example, the SMS/MMS application, the email application, the health care application, or the environment information application) to the external electronic device (for example, the electronic device 104). Additionally or alternatively, the notification relay application may, for example, receive notification information from the external electronic device (for example, the electronic device 104) and provide the received notification information to a user. The device management application may, for example, manage (for example, install, delete, or update) at least a part of functions of the external electronic device (for example, the electronic device 104) communicating with the electronic device 10 (for example, turn-on/turn-off of the external electronic device (or a part of its components) or control of the brightness (or resolution) of the display), an application executed in the external electronic device, or a service (for example, a call service or a message service) provided by the external electronic device. According to various embodiments of the disclosure, the applications 147 may include an application designated according to a property (for example, the type of the electronic device) of the external electronic device (for example, the electronic device 104). If the external electronic device is an MP3 player, the applications 147 may include an application related to music play. Similarly, if the external electronic device is a mobile medical device, the applications 147 may include an application related to health care. According to an embodiment, the applications 147 may include at least one of an application designed in the electronic device 10 or an application received from the external electronic device (for example, a server 106 or the electronic device 104).

The I/O interface 140 may provide a command or data received from a user or an I/O device (for example, a sensor, a keyboard, or a touch screen) to the processor 120, the memory 130, or the communication interface 160, for example, via the bus 110. For example, the I/O interface 140 may provide data about a user's touch received through the touch screen to the at least one processor 120. The I/O interface 140 may output a command or data received from the at least one processor 120, the non-transitory memory 130, or the communication interface 160, for example, via the bus 110 through the I/O device (for example, a speaker or a display). The I/O interface 140 may include an audio module, the audio module including hardware such as an integrated circuit.

The display 150 may display various types of information (for example, multimedia data or test data) to the user.

The communication interface 160 may establish communication between the electronic device 10 and an external device (for example, the electronic device 104 or the server 106). For example, the communication interface 160 includes hardware such as a transmitter, receiver, and/or transceiver, and a codec, and may be connected to a network 162 by wireless communication or wired communication and communicate with the external device over the network 162. The wireless communication may be conducted by, for example, at least one of wireless fidelity (WiFi), Bluetooth (BT), near field communication (NFC), GPS, or cellular communication (for example, long term evolution (LTE), LTE-advanced (LTE-A), code division multiple access (CDMA), wideband CDMA (WCDMA), universal mobile telecommunication system (UMTS), wireless broadband (WiBro), or global system for mobile communications (GSM)). The wired communication may be conducted in conformance to, for example, at least one of universal serial bus (USB), high definition multimedia interface (HDMI), recommended standard 232 (RS-232), or plain old telephone service (POTS).

According to an embodiment of the disclosure, the network 162 may be a telecommunications network. The communication network may include, for example, at least one of a computer network, the Internet, an Internet of Things (IoT), or a telephone network. According to an embodiment, a protocol for communication between the electronic device 10 and an external device (for example, a transport layer protocol, a data link layer protocol, or a physical layer protocol) may be supported in at least one of the applications 147, the API 145, the middleware 143, the kernel 141, or the communication interface 160.

According to various embodiments of the present disclosure, a controller may include the processor 120 and the memory 130 for storing information requested by the processor 120. The controller as a CPU provides overall control to the electronic device 10, and may also comprise additional integrated circuitry.

The electronic device 10 which will be described below may be any of the afore-described wearable device, laptop computer, netbook computer, smartphone, tablet PC, Galaxy Tab, and iPad. In the embodiment, the electronic device 10 may be a smartphone.

Further, the display of the electronic device may be configured to have a larger view area and an enhanced design by minimizing a bezel area. Or the display may be configured to be flexible, concave, or convex.

In other words, with regard to being flexible, since the periphery of the display is bendable, the view area may be extended to the side surfaces of the display. As a consequence, the view area may be extended or a separate screen may be used on a side surface of the display, and the display may be rendered to be sophisticated in design. In other words, the display includes a first view area and second view areas at both sides of the first view area.

According to various embodiments, the electronic device 10 may include a biometric information measuring apparatus, and the biometric information measuring apparatus may include a body temperature measuring apparatus and an ECG measuring apparatus. Besides a body temperature measuring apparatus and an ECG measuring apparatus, the biometric information measuring apparatus may be applied to other measuring apparatuses. For example, the biometric information measuring apparatus may include a photoplethymogram (PTG) measuring apparatus, a heart rate and blood pressure measuring apparatus, a blood glucose measuring apparatus, a galvanic skin response (GSR) measuring apparatus, and a body mass index (BMI) measuring apparatus. According to various embodiments of the present disclosure, the biometric information measuring apparatus will be described, taking a body temperature measuring apparatus and an ECG measuring apparatus are just a few non-limiting examples.

A configuration of a body temperature measuring apparatus (e.g. a contactless thermometer) 200 provided in the electronic device 10 according to various embodiments of the present disclosure will be described below in detail.

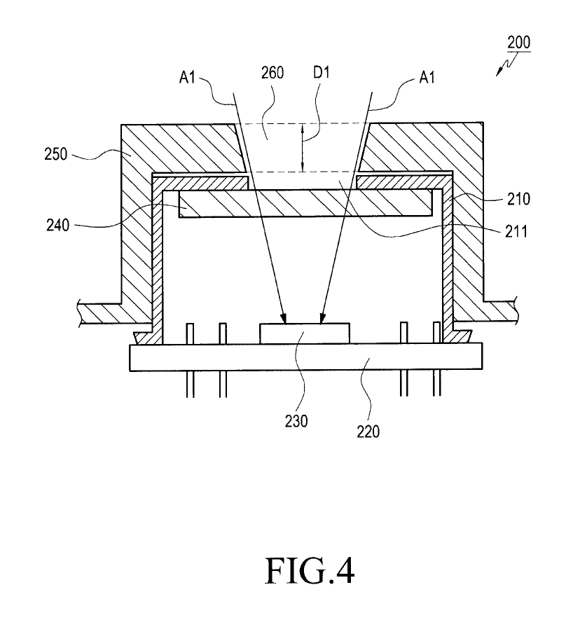

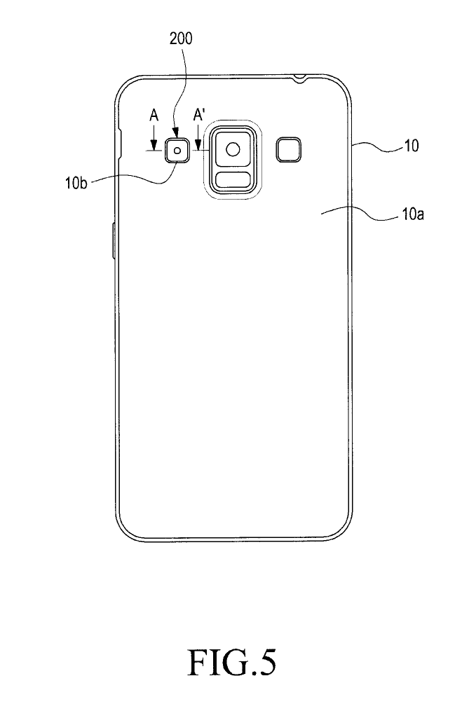

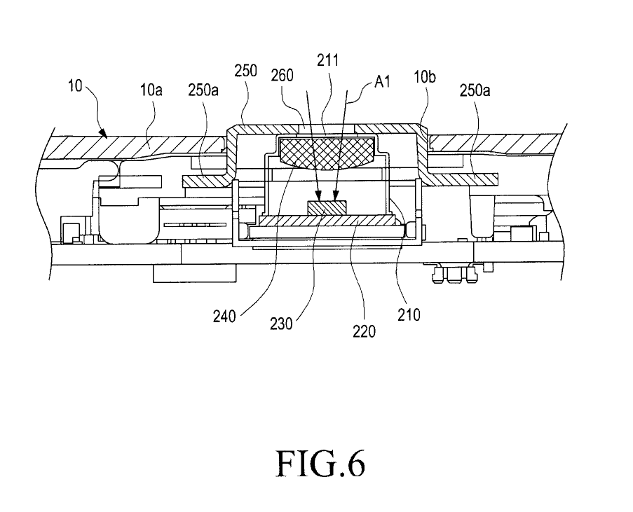

FIG. 4 is a side sectional view illustrating a structure of the body temperature measuring apparatus 200 according to various embodiments of the present disclosure, FIG. 5 is a front view illustrating the body temperature measuring apparatus (contactless thermometer) 200 provided on the rear surface of an electronic device according to various embodiments of the present disclosure, and FIG. 6 is a sectional view of the body temperature measuring apparatus 200, taken along line A-A′ illustrated in FIG. 5.

With reference to FIG. 4, the body temperature measuring apparatus 200 as one embodiment of the biometric information measuring apparatus will be described. The body temperature measuring apparatus 200 includes a cover 210, a printed circuit board (PCB) 220 having a sensor 230, a lens assembly 240, an electrode assembly 250, and a guide 260.

The cover 210 is provided with an opening 211 in order to face the later-described sensor 230.

The PCB 220 is disposed inside the cover 210 so that the sensor 230 may be positioned facing the opening 211.

The lens assembly 240 is disposed in the cover 211 to concentrate infrared (IR) light A1 generated from the user's body on the sensor 230 so that the sensor 230 may measure the body temperature of the user.

The electrode assembly 250 may include the guide 260 and may be disposed outside the cover 210 so that the guide 260 may face the opening 211.

The guide 260 faces the opening 211 to guide introduction of the IR light A1 and secures (provides) a measurement distance D1 for measurement of the body temperature of the user.

Since the electrode assembly 250 includes the guide 260. which guides introduction of the IR light A1 generated from the user's body and secures the measurement distance D1 for measurement of the body temperature of the user as described above, the measurement distance D1 may prevent a body temperature measurement error in spite of close contact between the electrode unit 250 and the user's body, and introduction of an error signal to the sensor 230, thereby increasing the accuracy of body temperature measurement of the product, compared to a conventional body temperature measuring apparatus that measures body temperature after a predetermined distance is secured between the cover 210 and the user's body.

The electrode assembly 250 may comprise an assembly of more than electrode, and may include an ECG electrode, a GSR electrode, and a BMI electrode. The electrode assembly 250 is described herein in the context of an ECG electrode, but is not limited to the ECG electrode. In other words, apart from the ECG electrode, the electrode assembly 250 may be applied to other electrodes.

Further, the electrode assembly 250 may be shaped into a cap so as to surround the periphery of the sensor 230. The electrode assembly 250 may be formed of a SUS-series metal. The electrode assembly 250 may be configured as a sensing pad.

Also, the guide 260 may have formed therein a trapezoidal guide hole that gets narrower from the outside of the electrode assembly 250 toward the inside of the electrode assembly. The guide 260 may be applied to a guide hole of any other shape. For example, the guide 260 may be shaped into the shape of a cylinder, a cone, an isosceles triangle, a rectangle, or a square.

The measurement distance D1 may be the thickness of a side of the electrode assembly 250. As illustrated in FIG. 4, the measurement distance D1 may span from the outside of the electrode assembly 250 to the opening 211 of the cover 210.

Referring now to FIGS. 5 and 6, the body temperature measuring apparatus (thermometer) 200 may be mounted in a mounting portion 10b formed on the rear surface of an exterior member 10a of the electronic device 10. Engagement members 250a are formed in the electrode assembly 250 to be fixedly caught by the inner surface of the exterior member 10a. More particularly, the electrode assembly 250 is mounted in the mounting portion 10b and at the same time, the engagement members 250a of the electrode assembly 250 are caught inside the mounting portion 10b.

The exterior member 10a may comprise any of a battery cover, a window, a touch panel, a front case, and a rear case. In the embodiment, the exterior member 10a will be described as a battery cover, by way of example.

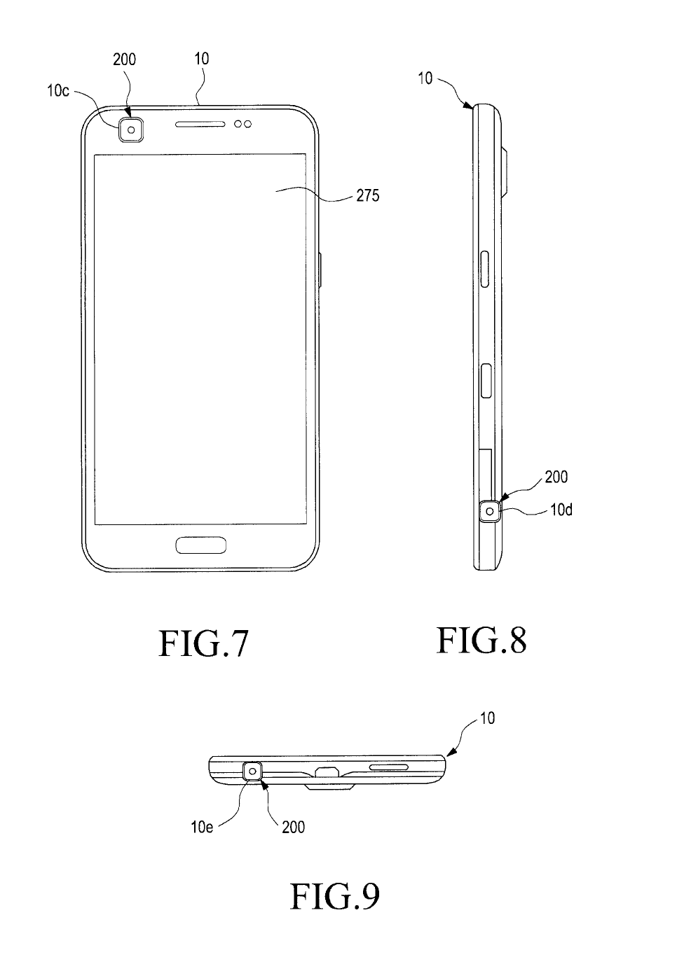

Also, the body temperature measuring apparatus 200 may be disposed at a different position of the exterior member 10a of the electronic device 10 than shown in FIG. 5. FIG. 7 is a front view illustrating the body temperature measuring apparatus provided on the front surface of an electronic device according to various embodiments of the present disclosure.

Referring now to FIG. 7, the body temperature measuring apparatus 200 may be mounted in a mounting portion 10c formed on the front surface of the exterior member 10a of the electronic device 10. More particularly, a display 275 is disposed on the front surface of the electronic device 10, and the mounting portion 10b is provided at a predetermined position around the display 275, for accommodating the body temperature measuring apparatus 200.

The body temperature measuring apparatus 200 may also be disposed at another different position of the exterior member 10a of the electronic device 10. FIG. 8 is a side view illustrating the body temperature measuring apparatus provided on one surface of an electronic device according to various embodiments of the present disclosure, and FIG. 9 is a side view illustrating the body temperature measuring apparatus provided on another surface (e.g. an end surface where the front and rear covers meet) of an electronic device according to various embodiments of the present disclosure.

Referring now to FIG. 8, the body temperature measuring apparatus 200 may be mounted in a first mounting portion 10d formed on one elongated lengthwise side surface (e.g. a lateral surface) of the exterior member 10a of the electronic device 10.

Referring now to FIG. 9, the body temperature measuring apparatus 200 may be mounted in a second mounting portion 10e formed on a surface perpendicular to the lengthwise direction.

Also, the body temperature measuring apparatus 200 may be accommodated selectively in the first or second mounting portion 10d or 10e.

More particularly, the first mounting portion 10d may be formed in at least a part of each of both elongated lengthwise side surfaces of the exterior member 10a of the electronic device 10. Therefore, the body temperature measuring apparatus 200 may be provided on each side surface of the exterior member 10a of the electronic device 10. Or the first mounting portion 10d may be formed on one side surface of the exterior member 10a, and the body temperature measuring apparatus 200 may be accommodated in the first mounting portion 10d. Likewise, the second mounting portion 10e may be formed on the bottom surface of the exterior member 10a of the electronic device 10, and the body temperature measuring apparatus 200 may be accommodated in the second mounting portion 10e. Or the second mounting portion 10e may be formed on each of the bottom and top surfaces of the exterior member 10a of the electronic device 10, and the body temperature measuring apparatus 200 may be accommodated in each of the second mounting portions 10e.

Further, the body temperature measuring apparatus 200 may be accommodated in each of the mounting portions 10b, 10c, 10d, and 10e formed respectively on the front, rear, and side surfaces of the exterior member 10a of the electronic device 10.

Also, the body temperature measuring apparatus 200 may be accommodated selectively in the mounting portion 10b, 10c, 10d, or 10e.

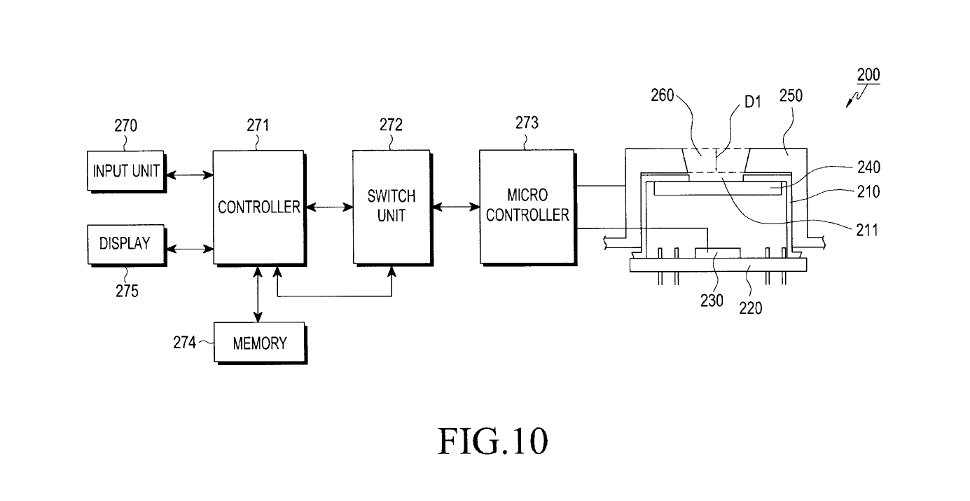

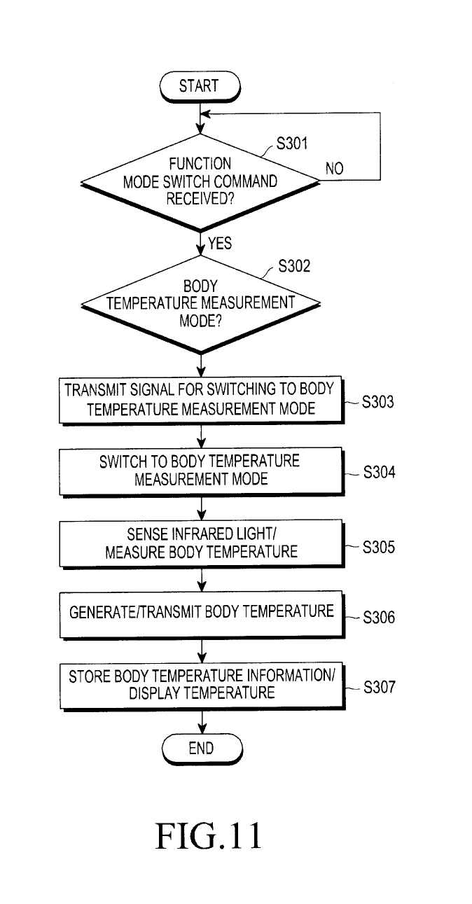

Now, an operation of the body temperature measuring apparatus 200 having the above-described structure in the electronic device 10 will be described in detail. FIG. 10 illustrates an operation of a body temperature measuring apparatus in an electronic device according to various embodiments of the present disclosure, and FIG. 11 is a flowchart illustrating a method for measuring body temperature by a body temperature measuring apparatus in an electronic device according to various embodiments of the present disclosure.

Referring now to FIGS. 10 and 11, the body temperature measuring apparatus 200 includes the cover 210 with the opening 211, the PCB 220 with the sensor 230, the lens assembly 240, and the electrode assembly 250 with the guide 260 that forms the measurement distance D1.

This body temperature measuring apparatus 200 is placed in the mounting portion 10b formed on the rear surface of the exterior member 10a of the electronic device 10.

In this state, a user contacts the body temperature measuring apparatus 200 mounted on the exterior member 10a on his or her body, and inputs a function mode switch command to an input unit 270 of the electronic device 10. At (S301), the input unit 270 transmits the function mode switch command received from the user to a controller 271.

At (S302, the controller 271 determines whether the function mode switch command is a command for switching to a body temperature measurement mode or to a general function mode.

If the controller 271 determines that the function mode switch command is a command for switching to the body temperature measurement mode, then at (S303) the controller 271 transmits a signal for switching to the body temperature measurement mode to a switching structure 272.

At (S304), the switching structure 272 switches to the body temperature measurement mode in response to the signal for switching to the body temperature measurement mode.

At (S305), the microcontroller 273 measures body temperature according to a signal corresponding to the IR light A1 sensed from the body of the user by the sensor 230 in the body temperature measurement mode.

As illustrated in FIG. 10, the microcontroller 273 is electrically connected to the sensor 230.

In other words, the electrode assembly 250 of the body temperature measuring apparatus 200 is brought into contact with the body of the user. The IR light A1 emitted from the user's body is introduced to the guide 260 and then transferred to the lens assembly 240 through the guide 260. The lens assembly 240 concentrates the IR light A1 onto the sensor 230 and the sensor 230 generates the signal corresponding to the IR light A1 by sensing the concentrated IR light A1.

At (S306), the microcontroller 273 measures body temperature according to the signal corresponding to the IR light IR, generates body temperature information about the measured body temperature, and transmits the body temperature information to the controller 271 using the switching structure 272.

At (S307), then the controller 271 stores the body temperature information in a memory 274 and displays a temperature indicated by the stored temperature information on the display 275 of the electronic device.

Thus, the user may determine the measured body temperature by viewing the temperature displayed on the display 275.

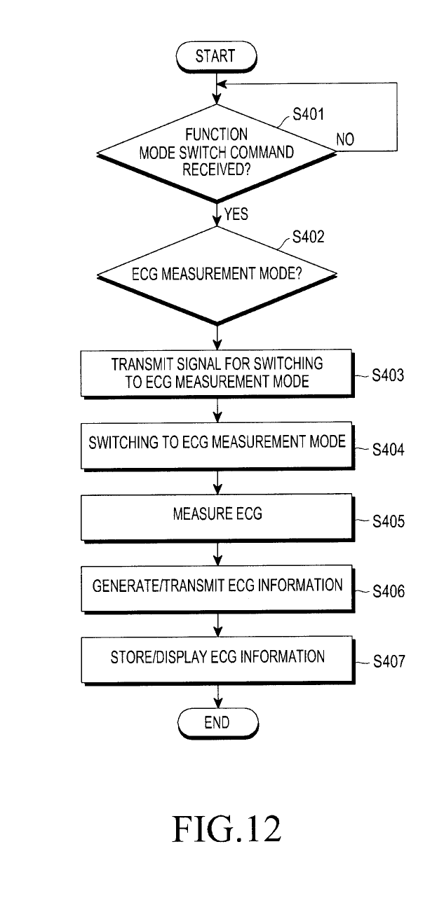

Another operation of the body temperature measuring apparatus 200 in the electronic device 10 will be described below in detail. FIG. 12 is a flowchart illustrating a method for measuring an electrocardiogram (ECG) in an apparatus for measuring body temperature provided in an electronic device according to various embodiments of the present disclosure.

The ECG short for electrocardiogram is a recorded curve of action current attributed to cardiac contraction. That is, myocardial excitement occurs in the venous sinus and advances toward the heart atrium/cardiac ventricle. The action current of the heart is illustrated as a graph by inducing the excitement to an ammeter (electrocardiograph) at two random points. Action potential generated from electrical excitement during myocardial activity is transferred to the surface of the body and recorded in the form of waveforms by current. This recording is the ECG. The ECG is used as very important data for diagnosing heart diseases.

As illustrated in FIG. 10, to measure an ECG, the body temperature measuring apparatus 200 includes the cover 210 with the opening 211, the PCB 220 with the sensor 230, the lens assembly 240, and the electrode assembly 250 with the guide 260 that forms the measurement distance D1.

This body temperature measuring apparatus 200 is placed in the mounting portion 10b formed on the rear surface of the exterior member 10a of the electronic device 10.

In this state, a user contacts the body temperature measuring apparatus 200 mounted on the exterior member 10a on his or her body, and inputs a function mode switch command to the input unit 270 of the electronic device 10. At (S401), the input unit 270 transmits the function mode switch command received from the user to the controller 271.

St (S402), the controller 271 determines whether the function mode switch command is a command for switching to an ECG measurement mode or a general function mode.

If the controller 271 determines that the function mode switch command is a command for switching to the ECG measurement mode, at (S403) the controller 271 transmits a signal for switching to the ECG measurement mode to the switching structure 272.

At (S404), the switching assembly 272 switches to the ECG measurement mode in response to the signal for switching to the ECG measurement mode.

At (S405), the microcontroller 273 measures an ECG from the user's body through the electrode assembly 250 including an ECG electrode in the ECG measurement mode.

The microcontroller 273 is electrically connected to the sensor 230.

In other words, the electrode assembly 250 of the body temperature measuring apparatus 200 is brought into contact with the body of the user. The electrode assembly 250 senses action current according to cardiac contraction of the user and generates a signal corresponding to the sensed action current.

At (S406), the microcontroller 273, which comprises of hardware such as integrated circuitry, is configured to measure the ECG according to the signal, generates information about the measured ECG, and transmits the ECG information to the controller 271 using the switching structure 272.

At (S407), the controller 271, which also comprises hardware such as integrated circuitry, stores the ECG information in the memory 274 and displays the stored ECG information on the display 275 of the electronic device.

Thus, the user may determine a cardiac state by viewing the ECG information displayed on the display 275.

Also, the electrode assembly 250 may have a transformer (not shown) to be isolated from an electrical signal (for example, a battery power supply signal, a transmission/received signal, and a ground (GND)).

Since the electrode assembly 250 contacts the user's body, the electrode assembly 250 should be isolated from an electrical signal of the electronic device 10 to protect the user from an electric shock.

Further, the electrode assembly 250 is isolated from an electrical signal of the electronic device 10 through the transformer, thereby preventing an electric shock that might occur when the user contacts the electrode assembly 250.

As is apparent from the foregoing description of the biometric information measuring apparatus according to various embodiments of the present disclosure, a measurement distance is secured by disposing an electrode assembly at the outermost side of a cover. Thus, as biometric information is measured with the measurement distance secured, introduction of a conventional error signal into a sensor and an error signal-incurring error in measuring body temperature can be prevented, thereby increasing the accuracy of measuring body temperature in a product. Also, conventional generation of electrostatic discharge (ESD) is prevented, thereby further increasing product performance.

Further, since a cap-shaped electrode assembly including electrodes for measuring an ECG, a GSR, and a BMI is configured, an ECG, a GSR, and a BMI as well as body temperature can be measured. As a consequence, utilization of the product can be increased.

The apparatuses and methods of the disclosure can be implemented in hardware, and in part as firmware or via the execution of software or computer code in conjunction with hardware that is stored on a non-transitory machine readable medium such as a CD ROM, a RAM, a floppy disk, a hard disk, or a magneto-optical disk, or computer code downloaded over a network originally stored on a remote recording medium or a non-transitory machine readable medium and stored on a local non-transitory recording medium for execution by hardware such as by at least one processor, so that the methods described herein are loaded into hardware such as a general purpose computer, or a special processor or in programmable or dedicated hardware, such as an ASIC or FPGA. As would be understood in the art, the computer, the processor, microprocessor, controller, control unit or the programmable hardware include memory components, e.g., RAM, ROM, Flash, etc., that may store or receive software or computer code that when accessed and executed by the computer, processor or hardware implement the processing methods described herein. In addition, it would be recognized that when a general purpose computer accesses code for implementing the processing shown herein, the execution of the code transforms the general purpose computer into a special purpose computer for executing the processing shown herein. In addition, an artisan understands and appreciates that a “processor”, “microprocessor” “controller”, or “control unit” constitute hardware in the claimed disclosure that contain circuitry, such as integrated circuitry, that is configured for operation. Under the broadest reasonable interpretation, the appended claims constitute statutory subject matter in compliance with 35 U.S.C. §101 and none of the elements are software per se. Nor are the claims directed to Abstract ideas.

The definition of the terms “unit” or “module” as referred to herein are to be understood as constituting hardware circuitry such as a CCD, CMOS, SoC, AISC, FPGA, at least one processor or microprocessor (e.g. a controller or control unit) configured for a certain desired functionality, or a communication module containing hardware such as transmitter, receiver or transceiver, or a non-transitory medium comprising machine executable code that is loaded into and executed by hardware for operation, in accordance with statutory subject matter under 35 U.S.C. §101 and do not constitute software per se.

While the disclosure has been shown and described with reference to certain exemplary embodiments thereof, it will be understood by those skilled in the art that various changes in form and details may be made therein without departing from the spirit and scope of the disclosure as defined by the appended claims and their equivalents.

What is claimed is:

1. An apparatus for measuring biometric information, the apparatus comprising:
a cover;
at least one sensor that measures biometric information provided inside the cover; and
an electrode assembly provided outside of the cover providing a measurement distance for measuring the biometric information.

2. The apparatus of claim 1, wherein the at least one sensor senses one of a body temperature, an electrocardiogram (ECG), a photoplethymogram (PTG), a heart rate, a blood pressure, a blood oxygen saturation, a heart rhythm, a respiration rate, and a blood glucose level of a user.

3. The apparatus of claim 1, wherein the electrode assembly includes a plurality of electrodes for respective measuring an ECG, a galvanic skin response (GSR), and a body mass index (BMI).

4. The apparatus of claim 1, wherein the apparatus for measuring biometric information includes one of a body temperature measuring apparatus, an ECG measuring apparatus, a GSR measuring apparatus, and a BMI measuring apparatus.

5. An apparatus for measuring body temperature, the apparatus comprising:
a cover having an opening;
a printed circuit board provided inside the cover and having a sensor facing the opening of the cover;
a lens assembly provided in the opening, for concentrating infrared light generated from the body of a user onto the sensor; and
an electrode assembly provided outside the cover,
wherein the electrode assembly includes a guide facing the opening of the cover, for guiding introduction of the infrared light and providing a measurement distance for measuring body temperature of the user.

6. The apparatus of claim 5, wherein the electrode assembly includes electrodes for measuring an electrocardiogram (ECG), a galvanic skin response (GSR), and a body mass index (BMI).

7. The apparatus of claim 5, wherein the electrode assembly is shaped into a cap surrounding a periphery of the sensor.

8. The apparatus of claim 5, wherein the electrode assembly is formed of an SUS-series metal.

9. The apparatus of claim 5, wherein the electrode assembly comprises a sensing pad.

10. The apparatus of claim 5, wherein the guide is a trapezoidal guide hole that becomes narrower from the outside of the electrode assembly to the inside of the electrode assembly.

11. The apparatus of claim 1, wherein the measurement distance is a thickness of a side of the electrode assembly.

12. An electronic device having an apparatus for measuring body temperature, the electronic device comprising:
a mounting portion provided on a rear surface of an exterior member of the electronic device;
a cover having an opening and being provided in the mounting portion;
a printed circuit board provided inside the cover and having a sensor facing the opening of the cover;
a lens assembly provided in the opening, for concentrating infrared light generated from the body of a user onto the sensor; and
an electrode assembly provided outside the cover,
wherein the electrode assembly includes a guide facing the opening, for guiding introduction of the infrared light and providing a measurement distance for measuring body temperature of the user.

13. The electronic device of claim 12, wherein the electrode assembly includes an electrocardiogram (ECG) electrode.

14. The electronic device of claim 12, wherein the electrode assembly includes an engagement member fixedly caught by an inner surface of the exterior member.

15. The electronic device of claim 12, wherein the exterior member comprises at least one of a battery cover, a window, a touch panel, a front case, and a rear case.

16. The electronic device of claim 12, wherein the apparatus for measuring body temperature is provided in a mounting portion formed on a front surface of the exterior member of the electronic device.

17. The electronic device of claim 12, wherein the apparatus for measuring body temperature is provided in a mounting portion formed on a side surface of the exterior member of the electronic device.

18. The electronic device of claim 12, wherein the apparatus for measuring body temperature is provided in a mounting portion formed on each of front, rear, and side surfaces of the exterior member of the electronic device, or the apparatus for measuring body temperature is provided selectively in the mounting portions.

19. The electronic device of claim 12, wherein the apparatus for measuring body temperature is provided in a first mounting portion formed on an elongated lengthwise side surface of the exterior member of the electronic device, the apparatus for measuring body temperature is provided in a second mounting portion formed on a surface perpendicular to a lengthwise direction, or the apparatus for measuring body temperature is provided selectively in the first or second mounting portion.

20. The electronic device of claim 12, further comprising:
an input for receiving a function mode switch command;
a controller including circuitry that generates a switching signal corresponding to the function mode switch command and controlling body temperature measurement and display by operating the electronic device in a body temperature measurement mode;
a switch that selectively actuates switching the electronic device to the body temperature measurement mode according to the switching signal;
a sensor that senses infrared light emitted from the body of the user that passes through the guide forming the measurement distance, and wherein the sensor generating a signal corresponding to the sensed infrared light;
a microcontroller including circuitry configured to measure a body temperature according to the signal corresponding to the infrared light and generating body temperature information corresponding to the measured body temperature;
a non-transitory memory for storing the generated body temperature information; and
a display that visually indicates a temperature based on the stored body temperature information.

21. The electronic device of claim 12, further comprising:
an input for receiving a function mode switch command;
a controller including circuitry configured to generate a switching signal corresponding to the function mode switch command and control ECG measurement by operating the electronic device in an ECG measurement mode;
a switch thatr switches the electronic device to the ECG measurement mode according to the switching signal;
an electrode assembly having an ECG electrode that contacts the body of the user and measures an ECG of the user;
a microcontroller including circuitry to measure the ECG of the user through the electrode assembly and to generate ECG information corresponding to the measured ECG;
a non-transitory memory for storing the generated ECG information; and
a display for displaying the stored ECG information.

22. The electronic device of claim 12, wherein the electrode assembly further includes a transformer for isolating the electrode assembly from an electrical signal generated from the electronic device.
